(19) United States
(12) Patent Application Publication
(10) Pub. No.: US 2022/0296363 A1
(43) Pub. Date: Sep. 22, 2022

(54) Accommodative Intraocular Lens Combination with Independent Fixed and Variable Power Lens Sections

(71) Applicant: AKKOLENS INTERNATIONAL B.V., Breda (NL)

(72) Inventors: Michiel Christiaan Rombach, Breda (NL); Willem Pieter Van Lawick, Breda (NL)

(21) Appl. No.: 17/636,460

(22) PCT Filed: Aug. 11, 2020
(86) PCT No.: PCT/NL2020/050505

§ 371 Date: Feb. 18, 2022

(30) Foreign Application Priority Data
Aug. 19, 2019 (NL) 2023662
Nov. 21, 2019 (NL) 2024278
Jan. 22, 2020 (NL) 2024728

(51) Int. Cl. A61F 2/16

(52) U.S. Cl. CPC A61F 2/1651; A61F 2/1635; A61F 2002/1682; A61F 2250/001

(57) ABSTRACT

The accommodative intraocular lens combination includes mechanically and optically independent lens sections including a static, fixed power lens section to restore refraction of the eye and an, independent, dynamic, variable power lens section to restore accommodation of the eye. The preferred embodiment is a combination of a fixed power lens section, for example, a monofocal intraocular lens implanted inside the capsular bag in combination with a variable power lens section implanted at the sulcus plane and driven by the ciliary mass directly. The lens can include optics comprising free-form surfaces according to orders which exceed third order Zernike and can include additional corrective optics to modulate fixed and variable residual optical.

### CROSS-REFERENCE TO RELATED APPLICATIONS

This application is the United States national phase of International Application No. PCT/NL2020/050505 filed Aug. 11, 2020, and claims priority to The Netherlands Patent Application Nos. 2023662 filed Aug. 19, 2019, U.S. Pat. No. 2,024,278 filed Nov. 21, 2019, and U.S. Pat. No. 2,024,728 filed Jan. 22, 2020, the disclosures of which are hereby incorporated by reference in their entirety.

### BACKGROUND OF THE INVENTION

### Field of the Invention

The invention relates to accommodative intraocular lenses and, particularly, to such lenses with fixed and variable lens sections.

### Description of Related Art

Accommodative intraocular lenses restore accommodation of the human eye, meaning, provide the retina with a sharp focus at any object distance, from far to reading distance, by translation of the focus the incoming light beam along the optical axis. Such accommodative lenses can vary the focal distance by movement of the lens along the optical axis, for example, movement of single fixed focus lenses in the eye, as disclosed in, for example, US2019053893 and WO2006NL50050 (EP1871299), or, alternatively, movement of multiple lenses along the optical axis, as disclosed in, for example, US2018221139 and US2013013060 (CA2849167, US2002138140). Such lens movements can be driven by the ciliary muscle, generally via the remains, via the rim, of the capsular bag, as in US2019053893, or, alternatively, such movement can be driven by the iris, as in, for example, WO2019027845, ES2650563 and US2008215146, or, alternatively, such movement can be driven by the zonulae which connect the capsular bag to the ciliary mass of the eye, as in, for example, US2018353288.

Alternatively, a single multifocal lens, for example, a monofocal lens, or, alternatively, a lens with a single cubic free-form surface or, alternatively, a lens with a bifocal or multifocal optical surface, can be moved in a direction perpendicular to the optical axis, as in US201010624.

All references to prior art documents mentioned in the present document are considered to be part of the present document.

Also, translation of the focus of a lens along the optical axis can be achieved by a change of lens shape, meaning: an elastical increase of radial thickness of the lens, along the optical axis, as in, for example, AU2014236688, US201562257087, US20190269500, U.S. Pat. No. 9,114,005 and US2018256315, which discloses lenses in which an elastic container filled with a fluid comprises the variable lens, or, alternatively, as in US2018344453, U.S. Pat. No. 10,004,595, US2018271645, US2019015198, U.S. Pat. Nos. 9,114,005 and 9,744,028 which disclose a change in shape of a uniform elastic lens and, alternatively, as in US2019000162 which discloses an elastic lens driven by fluid pressure of the vitreous of the eye. US2012310341, US2011153015 and DE112009001492 disclose any type of shape changing lenses which lenses are positioned at the sulcus plane, instead of inside the remains of the capsular bag of the eye with such change of shape driven directly by the ciliary mass or zonulae system of the eye, or, alternatively, by the iris, or, alternatively, by the sclera, for example by a sulcus extension connected to the sclera of the eye.

In addition, variable optics can be provided by two optical elements with each element comprising at least one free-form optical surface with such a shape that the combination of these shapes provides a variable lens of which the optical power depends on the relative position of the elements in a direction perpendicular to the optical axis, generally free-form shapes according to Louis Alvarez' basic patent U.S. Pat. No. 3,305,294, as in for example, EP1720489, with the optical elements connected by, for example, a mechanical connector, as in NL2015644, or by gluing, meaning: connection by a glue of which the material significantly differs from the material of which the lens is composed, or, alternatively, by re-polymerization, meaning: meaning: connection by material which does not significantly differs from the material of which the lens is composed. Such lenses can provide a variation on optical power in response to a change in the mutual position of the optical elements, as disclosed in, for example, NL2012133, with the said free-form optical surfaces distributed over any number of surfaces of the optical elements, as disclosed in, for example, NL2012420. Intraocular lenses comprising such free-form variable optics and their application are known from, for example, but not restricted hereto from, referring to such intraocular lenses and applications: WO2019022608 disclosing free-form surfaces of, for example, different Zernike orders, which algorithms can also be expressed by, for example, NURBS, or, alternatively, spline algorithms, or, alternatively, any other mathematical description for free-form surfaces. US2012323320 discloses such mechanically adjustable lenses; US2017312133 discloses such lenses which and adjustable by laser light; NL2015538 and US2014336757 disclose haptics for such lenses intended to be positioned at the sulcus plane; NL2015616 discloses irrigation channels for such lenses with the channels intended to increase intraocular flow of liquids to reduce increase in intraocular pressure; US2016030162 discloses an electricity generator driven by such lenses; WO 2009051477 discloses piggy back additional lens elements, meaning: thin lens elements added to main lens, generally on top of the main lens, with the elements providing correction of residual optical errors; US2014074233 and U.S. Pat. No. 9,744,028 disclose such lenses comprising additional anchoring components providing positioning of such lenses in the remains, for example the rim of the capsullorhexis, of the capsular bag; US2012257278 and EP1932492 disclose the principles of variable correction of any combination of variable aberrations; WO2014058316 discloses alternative shapes for the elastics haptics of such lenses; NL210980 and EP2765952 disclose customized optics of such lenses; NL2009596 discloses additional mechanical components to such lenses to protect the posterior surface of the iris of the eye.

The translation of the focus of a lens along the optical axis can be a parallel mutual shift of optical elements as used as the main example of variable lenses in this document, but also a rotation of at least one element as in the rotation of optical elements comprising at least two chiral optical surfaces in a direction perpendicular to the optical axis, WO2014058315 and ES2667277, or, alternatively, a combination of wedging and rotation of at least two complex free-form surfaces, for example adapted cubic optical surfaces, as in, for example, US2012323321, with such surfaces including, but not restricted hereto, Alvarez lenses composed of cubic free-form optical surfaces as disclosed in, or derived from, Louis Alvarez' basic patent U.S. Pat. No. 3,305,294.

All the references cited, as well as documents cited therein, are considered part of the present document.

So, all accommodative lenses including the lenses referred to above in the prior art comprise either (1) a single variable lens optical element providing a combination of fixed optical power to correct the refraction of the eye as well as variable optical power to correct accommodation, as in, for example, PT2775961 and other documents related to the same invention, or, alternatively, (2) comprise multiple dependent, meaning mechanically coupled, optical elements including an element for fixed optical power which element is mechanically coupled to and which drives a second optical element which provides the variable optical power, as in, for example, CN109806027 and other documents related to this invention.

The term ‘static fixed power lens section’, henceforth also: ‘fixed power lens section’ refers to both the mechanical properties as well as the optical properties of the fixed power lens section which section remains static, not moving, in the eye and which section provides to the eye at least one fixed optical power. The term ‘dynamic variable optical power lens section’, henceforth: ‘variable power lens section’ refers to both the mechanical properties as well as optical properties of the variable power lens section which the section is dynamic, moving, in the eye and which section provides variable optical power to the eye with the term ‘moving’ including translation of at least one component of the section as well as a curvature change of at least one component of the variable power lens section.

### SUMMARY OF THE INVENTION

This document discloses, firstly, accommodative lens combinations comprising at least two fully independent lens sections, with at least one fixed power lens section fully independent from the at least one variable power lens section with fully independent meaning optically and mechanically independent. So, the fixed power lens section provides emmetropia, as do current monofocal lenses, but the eye with only the fixed power lens section cannot accommodate. The variable power lens section provides accommodation, as do current accommodating intraocular lenses, but the variable power section does not provide the emmetropia. fixed power lens section. with fully independent meaning: optically independent and mechanically independent from the variable power lens section.

The ‘combination of lens sections’ refers to the combination of a fixed power lens section and a variable power lens section. A ‘section’ is composed of an optical lens, a fixed power lens or a variable power lens and a ‘section mechanical construction’ into which the lens is fitted. Such section mechanical construction can comprise ‘positioning means’, generally also referred to as ‘haptics’, which anchor an optical lens into the eye. ‘Movement transfer means’ are components which provide transfer and translate movement of ‘driving means’ into the eye, for example the ciliary mass of the eye to movement of the variable power lens or of specific components of the variable power lens.

The present document, the present invention, discloses an invention concerning an accommodating intraocular lens, henceforth: ‘lens’, comprising at least two independent lens sections, meaning: separate, non-connected, sections. The combination including at least one, static, fixed power lens section to provide restoration of refraction of the eye and at least one, dynamic, variable power lens section to provide restoration of accommodation of the eye. Note that the fixed power lens included in the fixed power lens section can function independently, in absence of the variable power lens, for example in case of a fixed power lens section which is a standard monofocal intraocular lens. Clearly, in such case the eye can focus, for example at far, by the fixed power lens section, but the eye cannot accommodate. Also, the variable power lens included in the variable power lens section can function independently, in absence of the fixed power lens section, for example in case of a variable power lens section implanted in an eye comprising the natural lens, for example, a presbyope natural lens, or any phakic eye. In such case the eye can focus at far, by the natural lens, and the eye can accommodate, by the variable power lens section.

### BRIEF DESCRIPTION OF THE DRAWINGS

FIG. 1 shows a schematic cross section of the human eye with the optical axis of the eye, 1, the cornea, 2, the anterior chamber of the eye, 3, in front the iris, and the posterior chamber of the eye, 4, behind the iris, 5, the sulcus, 6, the ciliary muscle/mass, 7, the zonulae connection the ciliary mass to the capsular bag, 8, the natural lens of the eye in the capsular bag, 9, representing a fixed power lens section, the retina, 10, and the optic nerve, 11. This figure also shows a variable power lens section, in this example a elastic lens section, 12, which lens changes optical power by change in at least one radius of at least one optical surface in a direction, 13, along the optical axis, driven by contraction or relaxation, perpendicular to the optical axis, of the ciliary muscle/mass of the eye in a direction, 14.

FIG. 2 (refer also to FIG. 1) shows the natural lens of the eye, in this example the natural lens representing the fixed power lens section, in combination with a variable power lens section, in this example a lens section comprising two optical elements, 15, which both translate in a direction perpendicular to the optical axis, 16, with elastic movement transfer means, 17, which means provide translation of the movement of the ciliary muscle/mass into a mutual, shift of the optical elements of the variable power lens section, which lens comprises, in this example, at least two free-form optical surfaces.

FIG. 3 (refer also to FIGS. 1-2) shows the preferred embodiment of the lens as set forth in the present document, with the lens comprising, firstly, a fixed power lens section positioned in the capsular bag, 18, from which the natural lens is removed thought the capsullorhexis, a hole, 19, and, secondly, a variable power lens section, 20, as also set forth in FIG. 2 of which the two elastic movement transfer means, 21, 22, each moving one optical element of the variable power lens section in a mutually opposite direction perpendicular to the optical axis.

FIGS. 4 and 5 (refer also to FIGS. 1-3) show an alternative embodiment with the natural lens of the eye, 23 (FIG. 4), representing the fixed power lens section and a fixed power lens section, in this example a monofocal lens implant, 28 (FIG. 5), in these examples in combination with a variable power lens section comprising two independent optical elements comprising free-form optical surfaces of which only one element, 24, comprises a rigid positioning means, 25, and a elastic movement transfer means, 26, (a combination of means for a single optical element as illustrated in, for example, WO2006NL50050, FIG. 7 and US2010106245, FIG. 2) which variable power lens section translates in a direction perpendicular to the optical axis, with the other element being a fixed power lens section, 27, in this example an optical surface added to the cornea by a, in this example, a cornea inlay, or, alternatively, a free-form surface enscribed/engraved into or onto the cornea by laser light.

Note that movement/translation perpendicular to the optical axis includes all such movements, including, but not restricted to, lateral translations, shifts, rotations, and wedgings, or any combination of such movements perpendicular to the optical axis.

FIG. 6 (refer also to FIGS. 1-5) shows an alternative embodiment of a lens with the fixed power lens section being a, for example, standard monofocal lens, 29, onto or into a free-form optical surface, for example, a cubic free-form surface/mask, 30, is fitted, which surface provides a lens of variable optical power of which the optical power depend on the mutual shift of the fixed power lens section and the variable power lens section in combination with the complementary free-form surface, 31, on the variable power lens section.

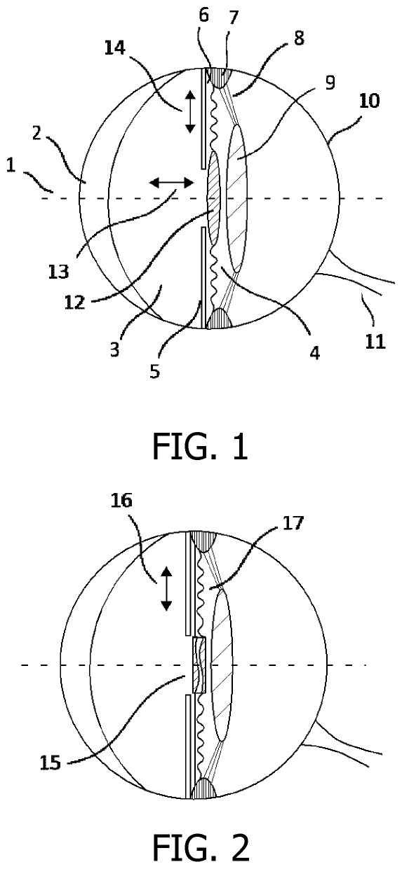

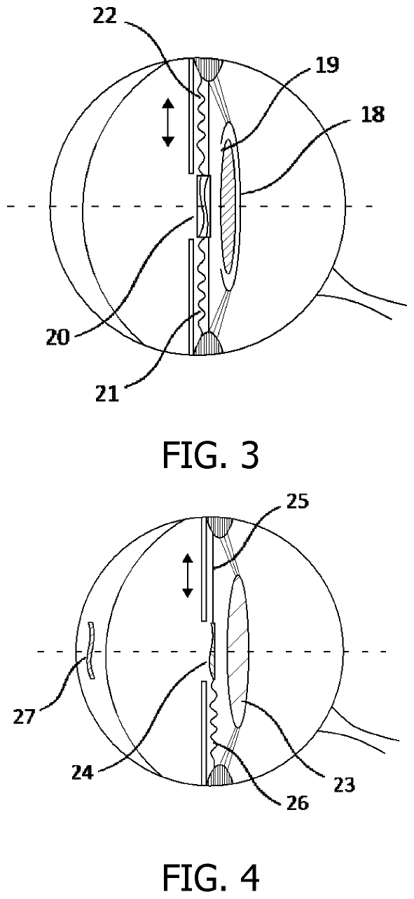

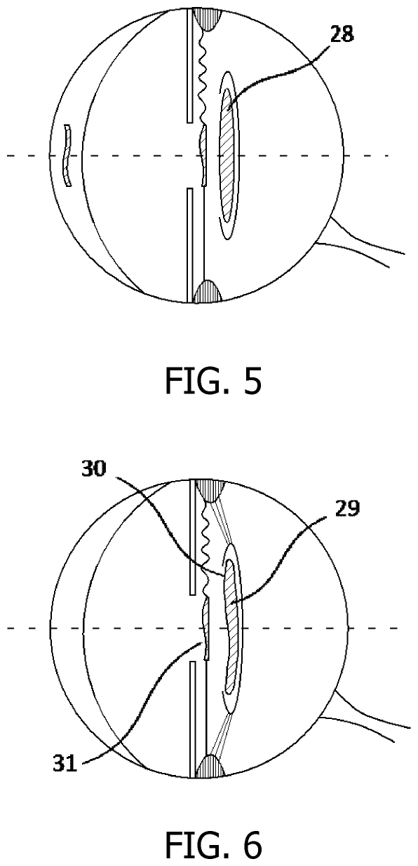

### DESCRIPTION OF THE INVENTION

The fixed power lens section, the refractive section, can comprise at least one optical component to provide fixed optical power to restore refraction of, for example, an eye, meaning an eye from which the natural lens is surgically removed, with the removal due a cataract of the eye or, alternatively, to clear lens extraction, CLE, meaning removal of a transparent lens due to, for example presbyopia and/or severe myopia. Note that the presbyopic, non-accommodating, natural lens of the eye can also be considered as a fixed power lens section.

Generally, the fixed power lens section is any artificial intraocular lens section implanted into the eye, for example, a monofocal intraocular lens, or, alternatively, a multifocal intraocular lens implanted in, for example, the capsular bag of the eye, or, alternatively, any lens implanted in any part of the eye, for example in the anterior chamber of the eye. Such fixed power lens section is generally implanted in the posterior chamber of the eye, in the remains, in the rim, of the capsular bag of the eye.

Such variable power lens section can comprise a combination of at least two optical elements comprising a combination of at least two free-form optical surfaces with each optical element comprising at least one free-form optical surface with the combination providing variable defocus power of which the power depends on the degree of mutual translation of the optical elements in a direction perpendicular to the optical axis of the eye as disclosed also in, for example, EP1720489. Such variable power lens sections comprise mechanical components to provide translation of, in this example, lateral compression of the section into mutual translation of the optical elements as disclosed in, for example, US2010106245 and multiple other documents cited therein and above in the present document.

A variable power lens section can also comprise at least one elastic optical component which component provides variable defocus power which power depends on the degree of change of shape of the elastic optical lens, a change of curvature, as disclosed in, for example, but not limited hereto, documents US2011153015 and US2019015198. Such variable power lens section also comprises a variable power lens section mechanical construction to provide transfer and translation by lateral compression of the movement transfer means into a change of shape of the elastic optical component. The elastic optical component can be made of uniform elastic lens material, as in, for example, DE11200900492 or, alternatively, can be uniform elastic material, for example, a fluid, into an elastic lens shaped flexible container or elastic lens shaped casing, as in, for example, but not restricted hereto, AU2014236688.

Variable power lens sections can be adapted to be implanted at the sulcus plane or ciliary plane of the eye, meaning in front of, anterior of, the capsular bag of the eye and can comprise at least one mechanical movement transfer means providing translation of movement of the ciliary mass or zonulae or any other related anatomical structure of the eye into mutual translation of the optical elements or, alternatively, in a change of shape of an elastic variable lens.

The lens combination can also comprise at least one additional optical surface to provide corrective optical power to correct at least one undesired optical aberration of the eye. For example, fixed optical power can correct a fixed power aberration, for example residual refractive error of the eye such as myopia, hyperopia or astigmatism of the eye, or any combination of such fixed power aberrations. Such corrective optics can be added to either the fixed power lens section or, alternatively, to the variable power lens section or, alternatively, the optics can be distributed over both sections. Alternatively, the variable power lens section can also comprise at least one additional optical surface to provide corrective optical power to enhance at least one desired variable optical aberration of the eye, in addition to the defocus aberration, which aberration can be a fixed optical power aberration or, alternatively, which enhancement of the other desired variable optical aberration can be, for example, enhancement of variable aspherical aberration to support sharp vision to support near, for example, reading, vision, or, alternatively, but not restricted hereto, variable aspherical aberration, or, variable astigmatism, or, variable coma, or, variable trefoil aberrations, or, any combination of any variable aberrations, with such variable corrections set forth in document EP193249.

So, the present document discloses a combination of an intraocular lens with a fixed power lens section and a variable power lens section which lens provides fixed optical power and variable power lens section providing variable optical power. The fixed power lens section provides at least a part of the fixed optical power of the lens and that the variable power lens section provides at least a part of the variable optical power of the lens. The fixed optical power restores the refraction of the eye to allow sharp vision at far and the variable power provides additional optical power to also allow the eye to focus at near distances, allows the eye to accommodate.

The present invention also relates to an accommodative intraocular lens combination with the lens combination comprising at least two lens sections including at least one fixed power lens section adapted to provide fixed optical power to the eye with the section comprising at least one fixed optical power lens fitted with at least one fixed power lens mechanical construction and with the combination including at least one variable power lens section adapted to provide variable optical power to the eye with the section comprising at least one variable power lens fitted with at least one variable power lens mechanical construction, wherein the first power lens section and the variable power lens section are not linked or connected, and preferably are, optically and mechanically, fully independent. The variable power lens mechanical construction and the fixed power lens mechanical construction may thus be separate constructions, wherein operation, movement or translation of one of the constructions does not influence the other, or cause the other the operate, move or translate as well.

The preferred embodiment of the combination includes a fixed power lens section by a monofocal intraocular lens implanted inside the capsular bag of the eye and a variable power lens section with variable power lens to provide variable optical power implanted outside, in front of, the capsular bag of the eye. Alternatively, another embodiment of the combination, includes a fixed power lens section by a monofocal intraocular lens implanted outside, in front of, the capsular bag of the eye and a the variable power lens section with variable power lens to provide variable optical power implanted inside the capsular bag of the eye. Again another embodiment of the combination includes a fixed power lens section by a monofocal intraocular lens implanted in the anterior chamber of the eye and the variable power lens section with variable power lens to provide variable optical power implanted in the posterior chamber of the eye, at the sulcus plane, or, alternatively, in the capsular bag, or, alternatively a combination of implant at the sulcus plane and in the capsular bag. Other embodiments can be designed as long as a driving component of the eye is, directly or indirectly, coupled to at least one movement transfer means of the variable power lens section, or, alternatively, with the movement transfer means of the variable power lens section mechanical construction directly, or, alternatively, indirectly, coupled an intraocular MEMS component.

The fixed power lens section can provide all fixed optical power of the lens with the variable power lens section providing all variable optical power of the lens, or, alternatively, the fixed power lens section can provide a part, the bulk, say, 18D, of the fixed optical power of the lens with the variable power lens section providing a limited part, say, 2D, of the fixed optical power of the lens. Generally it is advisable to add a limited optical power to any optical surface including surfaces of any lens system to prevent flat optical surfaces which flat surfaces can be undesirable because of scattering of the incoming light beam.

Note that flat surfaces on the variable power lens section can also be prevented by, for example, adding a weak spherical negative surface to the anterior surface of the section which a complementary weak positive spherical surface of an opposite sign on the posterior surface of the variable power lens section, or, alternatively, adding a weak positive spherical surface to the anterior surface of the section which a complementary weak negative spherical surface of an opposite sign on the posterior surface of the variable power lens section.

The lens sections can remain independent, meaning separate sections in the eye. However, the sections can also be mechanically coupled, in the eye, by any coupling component, for example, any pin-in-hole, groove-in-groove or other mechanical coupling means. Such coupling, preferably at the periphery of the mechanical constructions can rotational and tilt stability to the variable power lens section because the fixed power lens section is generally well stabilized inside the remains of the capsular bag by positioning means of the fixed power lens section.

The fixed power lens section can be a monofocal lens section implanted at any position in the eye, with the preferred position being apposition inside the remains of the capsular bag following explant of the natural lens of the eye. The fixed power lens section can comprise a single lens, for example a basic, traditional, spherical or aspherical lens, or, alternatively, a multifocal lens, for example a bifocal lens to provide at least one fixed optical power to provide restoration of refraction of an eye and the variable power lens section a single lens, for example, for example a basic spherical lens, or, alternatively, a multifocal lens, for example a bifocal lens to provide at least one fixed optical power with the combination of spherical lenses providing variable optical power of the intraocular lens.

The variable power lens section is a variable power lens section to provide all variable optical power, or, alternatively, part of the variable power, or, alternatively, the variable power lens section comprises at least one free-form optical surface which surface provides a variable lens in combination with at least one another such free-form surface which another free-form surface is not included in the variable power lens section, but, for example, is included as an optical components of the fixed power lens section, or, alternatively, is included in any other intraocular section, or, alternatively, is added, for example, onto or into the cornea of the eye by laser surgery or corneal implant.

The variable power lens section, the variable power lens section, can comprise a combination of at least two optical elements comprising a combination of at least two free-form optical surfaces with each optical element comprising at least one free-form optical surface with the combination adapted to provide variable defocus power of which the power depends on the degree of mutual translation of the optical elements in a direction perpendicular to the optical axis of the eye. Such accommodating lenses are known from, for example, EP1720489, NL2015644, NL2012133, NL2012420 and NL2009596 and many related documents hereto. The variable power lens section should also comprise mechanical components, haptics, adapted to provide translation of lateral compression of the section into mutual translation of the optical elements. The variable power lens section can also comprise at least one additional optical surface to provide corrective optical power to correct at least one optical aberration of the eye, for example, provide fixed optical power to correct at least one fixed optical aberration of the eye, which can be a residual refractive error of the eye, or, alternative, which can be myopia, hyperopia or astigmatism of the eye. Also, the additional optical surface provide variable optical power to correct at least one variable optical aberration of the eye other than variable defocus, for example, undesired variable aspherical aberration, or add the same, in case this is desired. The residual refractive error of the eye can be myopia of the eye, or, hyperopia of the eye, or, astigmatism of the eye, with additional optical surface providing variable optical power to correct at least one variable optical aberration of the eye other than variable defocus, for example, the variable optical aberration of the eye is variable aspherical aberration.

The shape of the posterior optical surface of the variable power lens section can be adapted to provide a fit with the anterior surface of the fixed power lens section to support proper movement of any component of the variable power lens section, or, alternatively, to prevent any movement, for example, decentration of the fixed power lens section. For example, a concave optical surface can be added to the posterior surface of the variable power lens section which surface can be compensated for a an added convex optical surface to the anterior surface of the fixed power lens section such that said surfaces provide support for centration of the variable power lens section versus the optical axis of the eye.

Such accommodating variable power lens sections are, preferably, implanted at the sulcus plane, or, alternatively, deeper in the sulcus of the eye, and driven directly by the ciliary mass/zonula system so that, with the section not present inside the capsular bag, posterior capsular opafication, PCO, or shrinkage of the capsular bag will not affect the accommodative properties of the lens section. Alternatively, the variable power lens section, the variable power lens section, can comprise at least one elastic optical component adapted to provide variable defocus power which power depends on the degree of change of shape of the elastic optical component.

Such components are known from AU2014236688, U.S. Pat. No. 1,011,745 and US2018256311, which documents disclose a lens shaped elastic container filled with a fluid or an elastic lens adapted to be implanted inside the remains of the capsular bag. US2019000612 discloses such lenses adapted to be implanted at the sulcus plane, in front of the capsular bag. So, the variable power lens section can comprise at least one elastic optical component which component provides variable defocus power which power depends on the degree of change of shape of the elastic optical component and comprises mechanical means, haptics, adapted to provide translation of lateral compression of the section into a change of shape of the elastic optical lens component. Such variable power lens sections are preferably implanted at the sulcus plane of the eye and comprise at least one mechanical movement transfer means providing translation of movement of any anatomical structure of the eye, for example the ciliary mass of the eye, into mutual translation of the two optical elements, or, alternatively, into a change of shape of the elastic optical component. So, the variable power lens section should comprises at least one mechanical component to provide translation of movement of the ciliary mass of the eye into mutual translation of the optical elements, or, alternatively, the variable power lens section should comprise at least one mechanical component to provide translation of movement of the ciliary mass of the eye into a said change of shape of the elastic optical component.

At least one of the lens sections can also comprises at least one additional optical surface provide corrective optical power to correct at least one residual optical aberration of the eye. Such corrections can be corrected for by the fixed power lens section, for example, severe corrections present in the eye pre-operative, for example, severe astigmatism due to cornea aberrations. Alternatively, corrections can be provided by the variable power lens section after implantation of the, likely large, fixed power lens section, of which the surgery can introduce additional aberrations of the eye. A method for such corrections is outlined in the section on methods outlined below in this document.

A method for implantation of a lens including a fixed power lens section and a variable power lens section with the fixed power lens section providing at least a part of the fixed optical power of the lens and the variable power lens section providing at least a part of the variable optical power. The procedure, the method, for implantation can be implantation of both the first and variable power lens sections during the same surgery. However, the method can also comprise multiple surgical steps including, firstly, replacement of the natural lens by a fixed power lens section, for example, a monofocal lens, as in, for example, standard cataract surgery, and secondly, after a period of time post operative, evaluation of residual fixed and variable aberrations of the eye and, thirdly, implantation of a second, customized, lens section adapted to provide a combination of accommodation and correction of any residual optical aberrations due to any optical characteristic of the particular eye and/or due to optical characteristic of the fixed power lens section and/or to the particular position to which the fixed power lens section has settled inside the eye. Such method can be designed to correct multiple residual refractive and other fixed optical aberrations and variable optical aberrations. The implantation of the variable power lens section is preferably before the corneal incision of the first implantation has fully healed so that no undesired aberrations are introduced by additional corneal incisions.

However, the optical functions of restoration of refraction of the aphakic eye and accommodation of the eye and correction of any residual optical aberrations can also be distributed over the fixed power lens section and the variable power lens section. Such distribution mainly applies to intraocular lens comprising a variable power lens section which section comprises combination of at least two optical elements comprising a combination of at least two free-form optical surfaces with each optical element comprising at least one free-form optical surface with the combination adapted to provide variable defocus power of which the power depends on the degree of mutual translation of the optical elements in a direction perpendicular to the optical axis of the eye.

For example, the fixed power lens section can comprises a combination of at least one optical component adapted to provide fixed optical power to provide restoration of refraction of an eye and at least one free form optical surface which, in combination with at least one complementary free-form surface provides a lens which provides variable defocus power of which the power depends on the degree of mutual translation of the optical elements in a direction perpendicular to the optical axis of the eye. Such fixed power lens section can be combined with a variable power lens section comprising one optical element comprising the complementary free-form surface. Such fixed power lens section can be implanted in a stable position in the eye, for example in the capsular bag, or, alternatively, in the anterior chamber, with a stable position meaning a position in which the section is intended not to translate. Or, alternatively, The fixed power lens section can comprise a standard monofocal lens, the variable power lens section a single free-form surface, with for example, a mechanical design as in EP1871299 and US2010106245 with the complementary free-form surface added on the cornea of the eye by, for example, a contact lens or into the cornea of the eye by, for example, a laser.

Or, alternatively, the variable power lens section can comprise two independent elements, firstly, a moving, translating, element comprising a free-form surface and a non-moving, static element comprising the complementary free-form surface. Such static element can be a piggy back element positioned on top of the fixed power lens section, or, alternatively, the static element can be the cornea of the eye on which a free-form surface is attached, for example by a contact lens or etched into the cornea by, for example, a laser, or, for example, such free-form can be etched on top of a anterior chamber intraocular lens. Or, alternatively, such free form can be added to the, preferably, anterior surface of any, static, intraocular lens, the fixed power lens section, inside the remains of the capsular bag. Such combination of two spherical optics of one of the optics translated in a direction largely perpendicular to the optical axis will result in a distorted image, by introduction of, for example, coma aberrations due to decentration. However, such aberrations can be minimized by concentration of the main fixed optical provided by the first, stable, lens section, for example, depending on the requirements of the particular eye, the fixed power lens section providing 20D of fixed optical power for refractive correction and the variable power lens section providing, say, 2.5D variable power for accommodation. With such combination the aberrations at accommodation might not be noticeable to the wearer of the intraocular lens.

The variable power lens section can comprises mechanical components to provide translation of lateral compression of the section into mutual translation of the optical elements, or, alternatively, the variable power lens section can comprise at least one elastic optical component which component is to provide variable defocus power which power depends on the degree of change of shape of the elastic optical component with the variable power lens section comprising mechanical components adapted to provide translation of lateral compression of the section into a change of shape of the elastic optical component, with the variable power lens section to be implanted at the sulcus plane of the eye with the variable power lens section comprising at least one mechanical component providing translation of movement of the ciliary mass of the eye into mutual translation of the optical elements, or, alternatively, the variable power lens section comprising at least one mechanical component providing translation of movement of the ciliary mass of the eye into a change of shape of the elastic optical component, and, the variable power lens section also comprising at least one additional optical surface adapted to provide corrective optical power to correct at least one optical aberration of the eye which can be fixed optical power to correct at least one fixed optical aberration of the eye, which can be residual refractive error of the eye, for example, myopia of the eye, or, hyperopia of the eye, or, astigmatism of the eye, or at least one additional optical surface provides variable optical power to correct at least one variable optical aberration of the eye other than variable defocus which can be variable aspherical aberration with the shape of the posterior optical surface of the variable power lens section providing a fit with the anterior surface of the fixed power lens section.

So, in summary, this document discloses an intraocular lens with a fixed power lens section and a variable power lens section with the lens providing fixed optical power and variable optical power with the fixed power lens section providing at least a part of the fixed optical power of the lens and that the variable power lens section providing at least a part of the variable optical power of the lens, or, alternatively, with the fixed power lens section providing all fixed optical power of the lens and that the variable power lens section provides all variable optical power of the lens.

The fixed power lens section can comprise a monofocal intraocular lens with the fixed power lens section implanted inside the capsular bag, and, the variable power lens section can comprise a variable intraocular lens with the variable power lens section implanted outside the capsular bag of the eye.

The variable power lens section can comprise a combination of at least two optical elements comprising a combination of at least two complementary free-form optical surfaces with each optical element comprising at least one such free-form optical surface with the combination adapted to provide a lens of variable defocus power which power depends on the degree of mutual translation of the optical elements in a direction perpendicular to the optical axis of the eye, or, alternatively, the variable power lens section can comprise at least one elastic optical component to provide variable defocus power which the optical power depends on the degree of change of shape of the elastic optical component.

Also, at least one of the lens sections can comprise at least one additional optical surface to provide correction of at least one residual optical aberration of the eye.

The method for implantation of such an intraocular lens can comprise multiple steps including, firstly, replacement of the natural lens by a fixed power lens section, and secondly, after a period of time, post operative, evaluation of residual fixed and variable aberrations of the eye and, thirdly, implantation of a variable power lens section adapted to provide a combination of accommodation and correction of any number of residual optical aberrations.

The intraocular lens and method for restoration of refraction and accommodation can provide for the following advantages over accommodative intraocular lenses as disclosed earlier in, for example, lenses comprising at least one single elastic optical element as in US2018344453, U.S. Pat. No. 10,004,595, US2018271645, US2019015198 and U.S. Pat. No. 9,744,028 which, and, EP1720489 and intraocular lenses comprising multiple, connected, optical elements as in as in NL2015644, NL2012133 and NL2012420, and, lenses referred to in any reference mentioned in this document and lenses related thereto referred to in any reference mentioned in this document.

Firstly, the first the fixed power lens section, implanted through a primary incision, in the capsular bag, providing at least a part of the fixed optical power, which can be the full fixed optical power to provide the full fixed optical power to correct for ametropia of the eye or part thereof, which fixed power lens section can be, for example, any standard spheric monofocal lens, or, alternatively, any aspheric monofocal lens, or, alternatively, any monofocal lens, or, alternatively, any monofocal lens providing also a toric correction, or, alternatively, any monofocal lens providing accommodation by a movement along the optical axis, or, alternatively, a monofocal lens providing a toric correction, or, alternatively, the fixed power lens section can be any multifocal lens, for example, a bifocal intraocular lens, a trifocal intraocular or any intraocular lens providing extended depth of field, meaning: any EDOF lens of any EDOF optical power which can be a lens of relatively weak EDOF optical power.

Secondly, the variable power lens section, providing at least a part of the variable optical power of the lens, implanted in front of the fixed power lens section as set forth above can be implanted during the same surgery during which the fixed power lens section is implanted. However, the variable power lens section can also be implanted at a later time, after the fixed power lens section is implanted, for example, 1-3 months later, preferably before the primary incision of the fixed power lens section implantation is healed, or even long after implantation of the fixed power lens section, which might be years after implantation of the fixed power lens section, which means that eyes comprising any intraocular lens can be implanted with a second lens section to provide accommodation to any such eyes comprising any intraocular lens in the capsular bag.

Such separation in time of implantation of the first and the variable power lens section provides the surgeon and patient for a full re-evaluation of vision. For example, the patient might be fully satisfied with the quality of vision and/or accommodation and/or EDOF provided by only the fixed power lens section and thus the implantation of the variable power lens section can be reconsidered, or, alternatively, the patient might be only partly satisfied with the vision and/or accommodation provided by only the fixed power lens section and requires an implantation of the second, accommodative, lens section, which variable power lens section can be customized according to such requirements, for example, customization of accommodative power and/or correction of any refractive error and/or any toric correction and/or correction of any other optical errors or addition of any other optical corrections all of which corrections can be fixed optical power corrections or, alternatively, variable optical power corrections.

Thirdly, the variable power lens section is a section of limited thickness because the fixed power lens section carries the main bulk, the main refractive lens. Such thin variable power lens section provides thus for a relatively simple explant, or, alternatively, exchange, of the variable power lens section in case the section does not provides the desired visual outcomes.

Fourthly, the method can include provision, by the variable power lens section, of a combination of accommodation and correction of any number of any residual optical aberrations which correction includes correcting for residual fixed refractive error, and/or correcting for residual variable refractive error, and/or correcting for any toric error and/or correcting for residual accommodative error.

Fifthly, the method provides for correction of the refraction of the eye by the implant of the fixed lens section which, at a later date, can be followed by the implantation of the variable power lens section. This allows the patient and doctor to decide to refrain from implantation of the variable power lens section in, the highly unlikely, cases the patient is fully satisfied by the optical performance of the fixed power lens section in combination with the, highly likely, use of spectacles for sharp near, reading, vision. So, the accommodative lens disclosed in the present document provides the patient and doctor with multiple choices to provide the patient with the most suitable and satisfactory outcome of the eye surgery.

So, the intraocular lens for restoration of refraction and accommodation as disclosed in the present document provides the surgeon and the patient a full choice of options regarding material, for example, hydrophobic versus hydrophilic materials, type, for example haptic section, and optical properties of the fixed power lens section, implanted in the bag, and, additionally, a similar full choice of options regarding the variable power lens section which section can be precisely customized to provide the final desired visual outcomes.

So, the present document discloses accommodative intraocular lens with the lens including at least two independent lens sections with the lens adapted to provide to the eye, with the eye having an optical axis, a combination of fixed optical power and variable optical power. The lens can comprise at least two independent lens sections including at least one fixed power lens section to provide at least one fixed optical power to the eye and at least one variable power lens section to provide variable optical power to the eye.

Note that the term ‘independent’ specifically excludes any other lens which are mechanically coupled, for example, a lens which provide driving the movement of a variable lens by a driving section, for example, the accommodating lens as disclosed in various documents from various sources, as in for example, U.S. Pat. No. 10,595,564, which discloses a ‘primary lens assembly’, the driving section, positioned in the capsular bag, into which, intraocularly, a ‘power changing lens’, the variable lens, is fitted and whereto the variable lens is coupled, which lenses are disclosed in various documents cited in the present document.

The fixed power lens section corrects, by adding fixed optical power to the eye, for the refractive error of the eye to which corrected eye the variable power lens section adds accommodation by adding variable power. So, the lens provides positioning in the eye of the fixed power lens section independent from the positioning of the variable power lens section in the eye. Note that a man skilled in the arts can conclude that the fixed power lens section can also be the natural, presbyopic, lens of the human eye to which only the variable power lens section is implanted, for example, implanted at the sulcus plane in front of the natural lens and behind the iris of the eye.

The fixed power lens section can comprise at least one monofocal optical component to provide monofocality to the eye, or, alternatively, the fixed power lens section can comprise at least one multifocal optical component to provide multifocality to the eye, or, alternatively, the fixed power lens section can comprise at least one EDOF optical component, for example a cubic optical surface or a pinhole type optical component to provide extended depth of field to the eye, or, alternatively, the fixed power lens section can comprise one combinatorial optical component which component to provide any combination of monofocality, multifocality and extended depth of field to the eye.

Alternatively, the capsular bag can remain empty with the fixed power lens section implanted in the anterior chamber of the eye with the variable power lens section implanted in the posterior chamber, for example, implanted in front of the empty capsular bag. Also, alternatively, the fixed power lens section can be split into at least two fixed power lens sections, with one implanted in the capsular bag and the other in the anterior chamber of the eye. Alternatively, the lens can comprises a fixed power lens section and a variable power lens section with both these sections positioned inside the capsular bag of the eye. Alternatively, the fixed power lens section can also be a variable power lens section which allows for implantation of multiple accommodative sections in the eye, for example, one variable power lens section inside the capsular bag and one variable power lens section in front of the capsular bag.

Note that at least one spacing component can be added to the accommodative lens combination with the spacing component providing separation of the variable and fixed power lens sections. Such component can be a separate component, or, alternatively, such component can be a component coupled to the variable power lens section, or, alternatively, to the fixed power lens section, or, alternatively, one such component can be coupled to the variable power lens section and one component to the fixed power lens section.

In the preferred embodiment the lens comprises a fixed power lens section which is fitted with at least one fixed power lens section haptic to provide anchoring of the fixed power lens section in the capsular bag of the eye, for example by traditional C-loop haptics or plate haptics or any other haptics which secure the section in the rim of the remains of the capsular bag from which the natural lens is removed through a capsullorhexis, a hole, in the anterior section of the capsular bag. Note that the fixed power lens section can comprise position means which are angled positioning means, in a direction parallel, along, the optical axis, providing positioning of the fixed power lens, for example, a lens inside the capsular bag, in a direction along the optical axis, for example, in a posterior direction along the axis which position further separates the variable power lens section and the fixed power lens section and thus prevents any mechanical interaction between these sections. Also, the variable lens section can be fitted with angled positioning means which, for example, position this section in an anterior direction along the axis which position further separates the variable power lens section and the fixed power lens section and thus prevent any mechanical interaction between these sections.

The variable power lens section can be fitted with at least one haptic which haptic is adapted to provide a combination of anchoring of the variable power lens section in front of the capsular bag of the eye and providing transfer of movement from any component in the eye to the variable power lens section. Such haptics are disclosed in multiple documents cited in the present document. The component in the eye can be a natural component, for example the ciliary muscle of the eye or any other anatomical structure in the eye, or, alternatively, such component can be a technical component, for example a microelectromechanical, MEMS, system to provide driving of the variable power lens section. So, the lens can comprises a variable power lens section comprising at least one haptic providing transfer of movement from any natural component in the eye to the variable power lens section, or, alternatively, the lens can comprise a variable power lens section comprising at least one haptic providing transfer of movement from any microelectromechanical system, MEMS, in the eye to the variable power lens section, or, alternatively, the lens can comprises a variable power lens section comprising at least one haptic providing transfer of movement from a combination of any at least one natural component in the eye and any at least one microelectromechanical system, MEMS, in the eye to the variable, for example, a MEMS which leverages, meaning: amplifies, movement of any natural component of the eye.

Any type of accommodative optics can be included in the variable power lens section, for example, an Alvarez variable lens as disclosed in U.S. Pat. No. 3,305,294, a variable lens comprising at least one combination of at least two optical elements each fitted with at least one free-form optical surface with the combination of the free-form optical surfaces providing a lens of variable optical power of which the degree of optical power depends on the degree of mutual translation of the free-form optical surfaces in a direction largely perpendicular to the optical axis of the eye.

Or, alternatively, as disclosed in documents cited in the present document, a variable lens comprising at least one elastic optical component which component providing variable optical power of which the degree of optical power depends on the degree of change of radial shape of the elastic optical component. Such radial elastic component can be a component comprised of one single flexible material, or, alternatively, multiple flexible materials, or, alternatively, a liquid filled container, an optic fluid chamber, as set forth in, for example, US2019358025.

Or, alternatively, a single piece accommodative optics, a multifocal lens, as in, for example, a bifocal lens as in WO2013105855, or, alternatively, a, linearly progressive, multifocal lens, an extended depth of field lens, for example, a cubic-PM phase mask as used for technical imaging as in, for example, WO9957599 and other documents related thereto and in WO2014135986 for ophthalmic applications. Note that a single free-form Alvarez optical surface as set forth elsewhere in this document is also a cubic phase.

Or, alternatively, a single largely spherical optical lens or two, optically relatively weak such lenses can function as accommodative optics. However, such optical constructions will inevitably lead to decentration of at least one optical component at at least one stage of the accommodative process thus resulting in undesired optical aberrations such as an optical coma of which the degree of aberration depends on the degree of shift of the optical element. However, such aberrations might be tolerable for the wearer of the leans because the fixed power lens section provides the bulk, say, 20D, of fixed optical power for refractive correction and the variable power lens section provides, say, only 2.5D variable power for accommodation.

Note that the free-form surfaces can be positioned on mutually connected optical elements, or, alternatively, can be positioned on independent optical elements, or, alternatively, can be at least one free-form surface fitted to a moving optical element with the other optical surface being a static optical surface, meaning: a, during accommodation, non-moving surface. A surface for example fitted to static optical element, for example a corneal inlay and any other intraocular implant, or, alternatively, can be a surface engraved by laser in the cornea of the eye.

The lens can include at least one additional optical surface on any surface and on any of the lens sections which additional surface provides optical correction of at least one residual fixed optical aberration of the eye, for example a surface to correct toric aberrations, a surface to correct for aspheric aberrations, or any surface to correct for aberrations of any Zernike order. Such surfaces can thus correct for any fixed optical aberration. Alternatively, two such, free-form, surfaces can correct for variable aberrations of any order, for example, correct for variable astigmatism or correct for variable asphericity or correct for multiple variable aberrations simultaneously. Note that such variable corrections can also provide for desired variable aberrations, for example desired aspherical aberration, as set forth in multiple document cited in the present document.

Any of the surfaces of the lens sections can be shaped to provide unhindered movement of at least one component of the variable power lens section. For example, the posterior surface of the variable power lens section can be of a concave to provide a maximum fit with a complementary convex shape of the anterior surface of the fixed power lens section to maximize positioning of at least one of the sections. Alternatively, the posterior surface of the variable power lens section can be of a convex to provide a minimum fit with a complementary convex shape of the anterior surface of the fixed power lens section. Alternatively, both surfaces can be planar with the desired optical power of the fixed power lens section concentrated on the posterior surface of the fixed power lens section. Such shapes depend on the properties of the lens materials which can differ between the variable power lens section and the fixed power lens section.

The preferred method for such accommodative intraocular lens, the method to maximize the desired optical performance in the individual eye, to fine-tune the optical properties of the lens to the specific optical requirements of the individual eye, can include, as in standard cataract or, alternatively, clear lens extraction surgery, firstly, removing the natural lens from the eye by, for example, phaco-emulsification, followed by, secondly, implanting the fixed power lens section, in the now eye, which lens implantation can be carried out by standard cataract procedures. The required implantation of the variable power lens section can be carried out at a later date, for example, after evaluation of any undesired residual aberrations of eye. So, optical corrections to correct for such undesired aberrations, for example, correct for undesired toricity or correct for refractive error for distance. Also, the variable power lens section can be fitted with additional optical surfaces to provide desired fixed aberrations, for example, desired fixed asphericity. or desired variable aberrations, for example desired variable asphericity. So, the lens can also provide to the eye with optical correction of any number of desired optical aberrations. Alternatively, the method can include implantation of the fixed power lens section followed directly, during the same surgery, by implantation of the variable power lens section in the eye. Alternatively, the method can include implantation of the fixed power lens section followed directly, during the same surgery, by implantation of the variable power lens section in the eye. Alternatively, the method can include evaluation of any undesired residual aberrations of the eye, meaning, in this example, the eye with a natural, for example presbyopic, lens followed by implantation of only the variable power lens section which variable power lens section also provides to the eye the optical correction of any number of undesired residual optical aberrations or adds to the eye any number of desired fixed optical corrections or variable optical corrections of which the concepts are further explained in this document or in references mentioned in this document.

So, the method can include providing a combination of accommodation and correction of any number of any residual optical aberrations which correction includes correcting for any toric error, or, alternatively, correction of any combination of accommodation and correction of any number of any residual optical aberrations which correction includes correcting for residual accommodative error, for example, a combination of accommodation and correction of any number of any residual optical aberrations which correction includes at least one correction of fixed optical power in combination with combination of accommodative power or any other variable correction of any variable aberration of any other variable aberration. Also, finally, any remaining variable and fixed aberrations can, of course, be corrected by intraocular laser treatment of the lens material as disclosed in, for example, U.S. Pat. No. 829,295, or, alternatively, by traditional modifications to the cornea optical surfaces by laser light, for example by lasik.

In summary, the present document discloses an accommodative intraocular lens, also ‘lens’, which includes at least two independent lens sections with the lens providing to an eye, having an optical axis, a combination of fixed optical power and variable optical power. The lens comprises at least one fixed power lens section to provide at least one fixed optical power to the eye and at least one variable power lens section providing variable optical power to the eye.

The fixed power lens section comprises at least one fixed power lens section haptic, one haptic, to provide anchoring of the fixed power lens section in the eye. The variable power lens section comprises at least one variable power lens section haptic which is adapted to provide anchoring of the variable power lens section in the eye and which haptic provides transfer of movement from any component in the eye to the variable lens, or, alternatively, the functions of anchoring the section and transferring movement to the variable lens are combined into at least one single haptic.

The variable power lens section can be manufactured from the same material as is the fixed power lens section. Alternatively, the variable power lens section can be manufactured from the same material, but with a different elasticity, for example, with a different water constant, as is the fixed power lens section. For example, the variable power lens section can be manufactured from, for example, a 18% hydrophilic acrylate which increase stiffness and allows for a thin lens design with the fixed power lens section manufactured from 26% hydrophilic acrylate which allows increased flexibility and thus injection of the lens through a relatively small incision in the eye. Alternatively, the variable power lens section can be manufactured from a different material as is the fixed power lens section, for example, one section of hydrophilic lens material and the section from hydrophobic material, or any lens section can be fitted with supporting component, any means, of again different materials, for example, and not restricted to this example, the fixed power lens section can be fitted with positioning means manufactured from PMMA.

The variable power lens section can comprise at least one combination of at least two optical elements each fitted with at least one free-form optical surface with the combination of the free-form optical surfaces to provide a lens section of variable optical power of which the degree of optical power depends on the degree of mutual translation of the free-form optical surfaces in a direction largely perpendicular to the optical axis of the eye, for example, largely cubic free-form optical surfaces. The translation of at least one of the free-form surfaces is, in general, a mutually opposite translation in a direction largely perpendicular to the optical axis of the eye but not restricted to only this direction.

So, such movement can be movement of at least one component of the variable power lens section in the direction along the optical axis, or, alternatively, movement of at least one component of the variable power lens section in the direction perpendicular to the optical axis, or, alternatively, a combination of said movements, or, alternatively, movement of at least one component of the variable lens in any direction. Alternatively, the variable power lens section can comprise at least one elastic optical component which component provides variable optical power of which the degree of optical power depends on the degree of curvature change in a direction along the optical axis of the elastic optical component. Such elastic optical component can be a component composed of a single, flexible material, or, alternatively, can be flexible container containing a fluid, or, alternatively, can be can be flexible container, the optical lens, containing a fluid with the container connected to supporting containers from which fluid is pumped to drive such optical lens, as disclosed in, for example, US2020000577 and documents related thereto. Furthermore, the variable power lens section can comprise one, largely, cubic free-form optical surface of which the degree of cubic curvature change depends on the degree of movement of at least one component of the variable power lens section mechanical construction.

The lens can include at least one additional optical surface adapted to provide optical correction of at least one residual optical aberration of the eye.

The lens can comprise a variable power lens section comprising at least one haptic adapted to provide transfer of movement from any at least one natural component in the eye to the variable power lens section, for example, from the ciliary muscle/mass of the eye. Alternatively, the variable power lens section can comprise at least one haptic adapted to provide transfer of movement from any at least one microelectromechanical system, MEMS, in the eye to the variable lens.

The preferred embodiment of the lens comprises at least two independent lens sections including at least one fixed power lens section, positioned inside the capsular bag of the eye, with the fixed power lens section to provide at least one fixed optical power to the eye to restore the refraction of the eye and with the lens including at least one variable power lens section, positioned in front of the capsular bag, at the sulcus plane to provide variable optical power to the eye to restore accommodation of the eye.

Note that any section, or both sections, of the combination can be fitted with any clamps or coupling components to provide coupling of the section to the rim of the capsullorhexis in the capsular bag to provide the section with increased positioning stability, as in, for example, US20120310341, or, alternatively, provide driving of the variable power lens by the rim the bag, as in, for example, WO2019235912.

The method for installing a lens disclosed above can include (1) removing the natural lens from the eye, (2) implanting the fixed power lens section into the eye, (3) evaluating any undesired residual aberrations of the eye and (4) implanting of the variable power lens section into the eye in which the variable power lens section which section also provides to the eye with optical correction of any number of undesired residual optical aberrations. Such method provides full control over the refractive outcomes, but requires a time period, for example a month, between the implantation of the first and the second lens section, resulting in two surgical interventions.

Alternatively, the method for installing a lens disclosed above can include (1) removing the natural lens from the eye, (2) implanting the fixed power lens section into the eye and implanting the variable power lens section into the eye during the same surgery. Such method provides less of an opportunity to fully control the refractive outcomes, but requires only a single surgical intervention.

Alternatively, in case the eye includes already an existing first lens section, for example, a presbyope natural lens, or a monofocal intraocular lens the method can include (1) evaluating of any undesired aberrations of eye and (2) implanting the variable power lens section into the eye with the variable power lens section providing accommodation to the eye and providing optical correction of any number of undesired optical aberrations. Such method provides full control the refractive outcomes, but requires only a single surgical intervention.

Alternatively, in case the eye includes already an existing first lens section, for example, a presbyope natural lens, or a monofocal intraocular lens the method can include implanting the variable power lens section into the eye which section to provide accommodation to the eye. Such variable power lens section can be a standard section which comprises no additional optical corrections. For example, an eye can be implanted with a standard monofocal lens leaving it open to the patient and the doctor to add a standard variable power lens section to provide accommodation as well at any time after the surgery.

Lastly, separate from the from the inventions disclosed above in the present document, the lens can comprises at least one fixed power lens section providing a combination of said fixed optical power and provide, in combination with at least one second lens section, said variable optical power. For example, the lens can comprise, firstly, a fixed power lens section in the capsular bag fitted with a single free-form surface, for example, on the anterior surface of the fixed power lens section and, secondly, the lens can comprise a variable power lens section fitted with a complementary single free-form surface, for example, on the posterior surface of the variable power lens section. The combination of free-form surfaces provide a variable lens, an Alvarez lens (lenses composed of cubic free-form optical surfaces as disclosed in, or derived from, U.S. Pat. No. 3,305,294), or such lenses as disclosed in multiple documents cited in the present document including references therein to other documents therein) of which the degree of optical power depends on the degree of mutual translation of said complementary free-form surfaces.

The accommodative intraocular lens combination comprises at least two independent lens sections including at least one, static, fixed power lens section to restore refraction of the eye and at least one, independent, dynamic, variable power lens section to restore accommodation of the eye. The preferred embodiment of the lens includes a fixed power lens section implanted inside the capsular bag, for example, any monofocal intraocular lens, and a variable power lens section implanted in front of the capsular bag, at the sulcus plane, with the sections separated in space, so, with the elements not being coupled. The lens can further include additional corrective optics to correct for both fixed and variable residual optical errors.

The haptics of the fixed power lens section can be implanted at an angle which angle differs from the angle with which the haptics of the variable power lens section are implanted, for example, a crosswise implant of said lens sections as to prevent interference of said haptics and to prevent stacking of haptics which might lead to an undesirable thickness of lens material which stacking can lead to, for example, a forward push of the iris of the eye which, in turn, can lead to narrowing of the anterior angle of the eye.

Also, a section of either the fixed power lens section or the variable power lens section can be fitted with at least one coupling component adapted to provide coupling of the section to the rim of the capsullorhexis in the capsular bag which coupling will, firstly, provide for stable positioning of the element and can, secondly, provide prevention of post operative rotation of the section as set forth in, for example, document WO2019235912, which prevention can be of importance for proper functioning of toric intraocular lenses.

The method can include implanting a lens combination, or section thereof, for example, only the variable power lens section, into a phakic eye, meaning: an eye comprising the natural lens, or, alternatively, the method can include implanting a lens combination, or specific sections thereof, into a phakic eye, meaning: an eye comprising not any lens, or, alternatively, the method can include implanting a lens combination into a pseudophakic eye, meaning: an eye comprising an artificial intraocular lens, for example, a monofocal intraocular lens but not restricted to only monofocal lenses.

This document discloses, secondly, optical principles which can be included in largely the variable power lens section of the accommodative lens combinations, meaning: the accommodative section of the accommodative lens combination. These inventions relate to an accommodating intraocular with at least two optical elements of which at least one element is adapted to move in at least one direction with optical surfaces providing variation of defocus aberration with the optical elements comprising at least one set of at least two free-form optical surfaces shaped according to a Zernike polynomial which order exceeds third order Zernike polynomials, exceed the order of the lens mentioned above, the Alvarez variable power lens according to Alvarez in U.S. Pat. No. 3,305,294 and documents related thereto. The following documents disclose aspects of such free-form optical surfaces exceeding a third order Zernike polynomial with US-A-2015/0342728 (D1), WO-A-2010/131955 (D3), NL-A-2012133 (D4), and EP-A-1 932 492 (D2) disclosing the IOL correction surfaces of higher orders including embodiments of fourth order free-form surfaces in combination with third order free-form surfaces. NL-A-2012420 (D5) and NL-A-2012420 (D5) relate to the distribution of the optical surfaces over the different available optical elements. Note that such optical principles can also apply to any variable lens designs, including lenses of any shape shifting, variable radius, design as mentioned in the first section of the present document. WO-A-2009/051477 is the application relating to the location of the lens in the sulcus of the eye and NL-A-2009596 relates to the mechanical construction of the IOL and the haptics.

Such accommodating, AIOL's, with at least two cubic free-form surfaces are well known from other documents, for example from NL-2012133, NL-201242, EP1871299, EP1932492. Designs of such AIOLs, and clinical results are disclosed in, Alio et al, Am. J. Ophthamol. 2016 April, 164: 37-48.

However, these AIOLs are restricted to the concept which include at least two cubic optical surfaces, that are optical surfaces including Zernike third order surfaces, of which the basic, free-form, non-rotational symmetric, shapes are known from Alvarez U.S. Pat. No. 3,305,294, which provides the original concept for a variable lens, for laterally shifting optical elements, and Baker CA-1252655, for derived fan-like rotational shifting optical elements. These are all various variations on the formula according to Alvarez, t=A(xy2+x3/3), see U.S. Pat. No. 3,305,294 for details and notations). Note that all these documents refer to only the Zernike third order formulas.

The optical surfaces disclosed in the present document comprise at least one set of at least two free-form, meaning: rotational asymmetrical, optical surfaces with these surfaces shaped according to a Zernike polynomial which order exceeds third order Zernike polynomial (as in said Alvarez U.S. Pat. No. 33,052,294) with at least one such surface fitted to each optical element. One of the present preferred embodiments comprises for example four surfaces each fitted with a fourth order polynomial surface with each element of the lens fitted with two such surfaces, one surface fitted to each side of the element. Said optical surfaces can include at least one optical shape according to a polynomial of the fourth degree, for example, in the most basic form, without any additional terms: z=S₀(x,y)=A(x⁴/4+xy³), with z being the sag of the optical surface, with x and y the values of X,Y coordinates presenting the plane perpendicular to the optical axis, the Z coordinate, and with A being a constant. Said optical surfaces can be arranged such that the arrangement a third order optical function, as in: S₁=S₀(x+Δx,y) and S₂=S₀(x+Δx,y) which a combination results in S₁−S₂≈2AΔx(x³+y³) which optical function provides said variable optical power.

Said optical surfaces shaped according to a Zernike polynomial which order exceeds third order Zernike polynomial which set provides variable focus, defocus, can be combined with additional sets of optical surfaces of any Zernike order to variably correct for any other optical aberration, for example, for variable optical cylinder, for variable optical coma or for variable optical aspherical aberration. lens fitted with two such surfaces, one surface fitted to each side of the element. Said optical surfaces can include at least one optical shape according to a polynomial of the fourth degree, for example, in the most Such lenses can also provide, by movement of at least one of the optical elements, a variable extension of accommodation, and/or variable focus, and/or variable spherical aberration. Such movement can be achieved, but not restricted to, principles as disclosed in US-2009062912 and WO-2005084587, and the same concept, with various adaptations in, for example, US-2014074233, WO-2014058316, EP-2765952, NL-2012257278, US-2010131955, US-2010106245, NL-1029548 and references made therein and related documents, which principles have been shown to function when fitted to an accommodating intraocular lens in the human eye.

Alternatively, such lenses can incorporate at least two optical elements with each element with at least one optical surface with an optical shape according to a polynomial of the fourth degree. Said optical surfaces can be distributed over any combination of anterior sides and posterior sides of said optical elements in an arrangement to provide a third degree optical function, also: a third degree wavefront. The optics of the lens construction, for example, the set of fourth order optical surfaces, can be adapted to provide correction for accommodation, meaning: providing variable defocus to correct for defocus aberrations of the eye.

The translation of at least one of the optical elements of the variable power lens is, generally, a shift, meaning: sliding, of the element in a direction perpendicular to the optical axis, as set forth in, for example WO2005084587, or, alternatively, a partial rotation in a plane perpendicular to the optical axis, as set forth in CA1252655, or, alternatively, a wedge shaped movement, that is a combination of a rotation and a translation of the optical elements in a plane largely perpendicular to the optical axis, or, alternatively, any combination of any movements movement in a plane largely perpendicular to the optical axis.

The lens construction can comprise at least one anchoring haptic, meaning: a mechanical component adapted to provide positioning and anchoring of the optical elements in the anterior chamber or the posterior chamber of the eye. Such haptics are included in almost every intraocular lens, ‘IOL’, which haptics can be for example plate haptics or C-loops for monofocal IOLs and multifocal IOLs, which are IOLs which do not require any movement in the eye. Examples of haptics translating a component of the IOL, AIOL, are the elastic loops as in WO-2005084587.

Hence, the present lens construction should comprise at least one translation haptic, meaning: a mechanical component adapted to provide, by coupling to translation of at least one optical element by transfer of movement of at least one component in the eye, a component preferably related to accommodation, to at least one of the optical elements. For example, the construction can comprises at least one haptic coupled to a natural component of the eye which component is the ciliary mass of the eye, as in WO-2005084587, or, alternatively, at least one haptic is coupled to a natural component of the eye which component is the capsular bag of the eye, or, alternatively, at least one haptic is coupled to a natural component of the eye which component is the zonula network of the eye, or, alternatively, at least one haptic is coupled to a natural component of the eye which component is the iris of the eye as in WO-2007027091, or, alternatively, at least one haptic is adapted to translate at least one of the optical elements by liquid pressure generated in the posterior chamber of the eye, as in, for example, HK-1066160, or, alternatively, an flexible optical element which changes shape by infusion of liquids from containers coupled to the optical element, as in for example US-2011282443, or, alternatively, any combination of means of translation including but not restricted to the examples cited above.

Also, at least one haptic of the present invention can be coupled to a MEMS, meaning: micro-electro-mechanical system, which MEMS is adapted to provide movement of at least one optical element. Such movement can adjust at least one of the elements to a fixed position, fixed endpoint, for example, a fixed resting position to adjust emmetropia of the eye, or, alternatively, set fixed positions for the range of accommodation, or, alternatively, provide leverage for accommodative power driven by ciliary muscle contractions or by external signals, for example signals from a smartphone. To illustrate this concept: the phone can have three buttons marked ‘far vision’, ‘intermediate vision’ and ‘reading’. The accommodation could also be driven by signals from brain waves of the wearer of the lens which sounds farfetched, but which concept is well known to a man skilled in the art.

The power for such MEMS can be supplied by an electric power generator, being a combination of at least one micro-magnet and one micro-coil which generates electric current during accommodation of the eye, or, alternatively, a micro-coil which generates current by external power sources such as a dedicated external magnetic wave generator, as in, for example, WO-2017039672 or, alternatively, by power generated by mobile telephones.

The lens construction can comprise at least one single circulating oblong flexible haptic which is adapted to change shape when the driving means are active such that the ratio of the length of the chief axis and the length of the transverse axis of said haptic decreases when the driving means are active with said ratio increasing when the driving means are inactive. The lens construction can comprise at least one single circulating oblong flexible haptic, as in, for example, WO-2014058316, which is adapted to change shape when the driving means are active such that the ratio of the length of the major axis and the length of the transverse axis of said haptic decreases when the driving means are inactive with said ratio increasing when the driving means are active.

The lens construction comprises at least one combination of connection points which combination comprises at least one optics connection point and at least one driving connection point, both connected to the haptic at the point where the chief axis of the haptic transverses the transverse axis with said combination adapted to provide translation of movement of driving means into movement of at least one optical element along the chief axis.

The lens construction comprises at least one combination of connection points which combination comprises at least one optics connection point and at least one driving connection point, both connected to the haptic at the point where the chief axis of the haptic transverses the transverse axis with said combination adapted to provide translation of movement of driving means into movement of at least one optical element along the transverse axis.

The lens construction comprises at least one haptic adapted to urge the optical element back to a resting position, a position of decreased optical power, when the driving means are inactive, or, alternatively, the lens construction can comprise at least one haptic adapted to urge the optical element back to a resting position, a position of decreased optical power, when the driving means are active.

The optical elements can also comprise at least one fixed power optical surface to correct for any fixed optical disorder of the eye, for example, correct for presbyopia, also: reading far-sightedness, or, alternatively, correct for myopia, near-sightedness, or, alternatively, correct for variable disorder is a variable disorder generated by the lens construction, or, alternatively, is adapted to provide correction of any combination of disorders of the eye, or, alternatively, the lens construction can comprise an additional fixed power optical element which provides fixed optical power and at least one optical element which element is a multifocal lens, providing at least two distinct foci, which lens is adapted to provide different optical powers at different relative positions in a plane perpendicular to the optical axis, or, alternatively, the lens construction can comprise a pinhole-component adapted to provide extended depth of field. The lens construction can be adapted to provide correction of any combination of variable and fixed disorders of the eye.

The ciliary muscle of the eye pulls the, gel-like, natural lens of the eye to a flattened shape to focus the eye at distance. Once the ciliary muscle relaxes, to focus the eye at closer distance, the natural lens regains its shape, the resting state, by the elasticity of the natural lens. So, the mechanism for a lens construction as presented in this document can be as follows: —at least one of the translation haptics of the lens can be fitted, at manufacturing, with at least one suture adapted to fixate the translation haptics of the lens in a compressed shape, meaning: a shape resulting in an increased optical power, —the suture is released by any releasing means after implant in the eye, meaning: released post-op, after the peripheral remnants of the capsular bag have fused with the haptics, generally after approximately a month post-op, fusion with said haptic or dedicated part thereof, —for vision at distance the bag expands, flattens, by natural means, meaning: relaxation driven by the ciliary muscle and zonulae which connect the bag to the ciliary mass, and, for vision closer, the ciliary muscle/mass contracts and the lens construction regains its resting state, meaning an optical power for close vision, while also pulling the bag inwards because of the fusion between haptic and bag. The suture can be adapted to be released by mechanical means, meaning: releasing by surgery, or, alternatively, the suture is adapted can be released by optical, meaning: releasing by laser light, by laser suturolysis, for example by laser light suitable to affect a vicryl material suture. Such fusion of bag and lens is known from prior art, U.S. Pat. No. 11,562,035 and US-20040243233A1, but only for a different mechanical concept, restricted to expansion of the bag, and not for contraction of the bag, which is a novel concept disclosed in the present document.

So, in summary, the present document discloses an accommodating intraocular lens construction, lens adapted to be implanted in the human eye, inside the capsular bag of the eye, or, alternatively, at the sulcus plane of the eye in front of the capsular bag, or, alternatively, at any location in the eye with the lens having an optical axis with the construction comprising at least two optical elements of which at least one element is adapted to move with the optical surfaces adapted to vary at least one optical aberration of the lens with a degree of variation which is dependent on the degree of movement of the at least one of the optical elements, which movement can be in at least one direction largely perpendicular to the optical axis, with the construction comprises a set of at least two free-form, meaning: rotational asymmetrical, optical surfaces with these surfaces shaped according to a Zernike polynomial which order exceeds any third order Zernike polynomial, with at least one such surface fitted to each optical element, with the set of optical surfaces providing correction for accommodation, meaning: variable defocus aberration, of the eye and with the translation of at least one of the optical elements is a shift, meaning: sliding, of the element in a direction perpendicular to the optical axis or the translation of at least one of the optical elements a rotation in a plane perpendicular to the optical axis which can be a translation of at least one of the optical elements is wedging in a plane largely perpendicular to the optical axis, or, a translation of at least one of the optical elements is any combination of any movements movement in a plane largely perpendicular to the optical axis, with the construction comprising at least one anchoring haptic, meaning: a mechanical component adapted to provide positioning and anchoring of the optical elements in the anterior chamber or the posterior chamber of the eye or the construction comprising at least one translation haptic, meaning: a mechanical component adapted to provide translation of at least one optical element by transfer of movement of at least one component in the eye to at least one of the optical elements, or, the construction comprising at least one haptic adapted to provide a combination of positioning and translation, with at least one haptic is coupled to a natural component of the eye which component is the ciliary mass of the eye, which is a natural component of the eye which component is the capsular bag of the eye, or, the zonula network of the eye, or, the iris of the eye, or, liquid pressure generated in the posterior chamber of the eye, or, a MEMS, meaning: micro-electro-mechanical system, which MEMS is adapted to provide movement of at least one optical element, or, with the lens construction comprises at least one single circulating oblong flexible haptic which is adapted to change shape when the driving means are active such that the ratio of the length of the chief axis and the length of the transverse axis of said haptic decreases when the driving means are active with said ratio increasing when the driving means are inactive, or, with the lens construction comprises at least one single circulating oblong flexible haptic which is adapted to change shape when the driving means are active such that the ratio of the length of the major axis and the length of the transverse axis of said haptic decreases when the driving means are inactive with said ratio increasing when the driving means are active, or, with the lens construction comprises at least one combination of connection points which combination comprises at least one optics connection point and at least one driving connection point, both connected to the haptic at the point where the chief axis of the haptic transverses the transverse axis with said combination adapted to provide translation of movement of driving means into movement of at least one optical element along the chief axis, or, that the lens construction comprises at least one combination of connection points which combination comprises at least one optics connection point and at least one driving connection point, both connected to the haptic at the point where the chief axis of the haptic transverses the transverse axis with said combination adapted to provide translation of movement of driving means into movement of at least one optical element along the transverse axis with the lens construction comprises at least one haptic adapted to urge the optical element back to a resting position, a position of decreased optical power, when the driving means are inactive, or, that the lens construction comprises at least one haptic adapted to urge the optical element back to a resting position, a position of decreased optical power, when the driving means are active, with, in addition that the optical elements also comprise at least one optical surface to correct for any fixed optical disorder of the eye, with the fixed optical disorder being presbyopia, also: reading far-sightedness, with the lens construction adapted for implant in the human eye to correct for at least one variable optical disorder of the eye with the variable disorder being a variable disorder generated by the lens construction, or, correction of any combination of at least one variable and at least one fixed disorder of the eye, with, alternatively, for implantation in the capsular bag the haptics are fitted, at manufacturing, with at least one suture adapted to fixate at least one of the translation haptics of the lens in a compressed shape, meaning: a shape resulting in an increased optical power, with the suture is adapted to be released by any releasing means after implant in the eye, meaning: released post-op, with the suture is adapted to be released by mechanical means, meaning: releasing by surgery, or, with the suture is adapted to be released by optical, meaning: releasing by laser light, by laser suturolysis, with preferably the suture is a vicryl material suture.

Also, an accommodating intraocular lens construction, lens adapted to be implanted in the human eye, with the lens having an optical axis with the construction comprising at least two optical elements of which at least one element is adapted to translate in at least one direction largely perpendicular to the optical axis with the optical surfaces adapted to vary at least one optical aberration of the lens with a degree of variation which is dependent on the degree of shift of the at least one of the optical elements can be a construction comprises a set of at least two free-form, meaning: rotational asymmetrical, optical surfaces with these surfaces shaped according to any Zernike polynomial of any order, including 3rd order Zernike polynomials.

Such accommodating intraocular lens construction according can be fitted with at least one flange which is fitted to the posterior optical element, which flange is adapted to provide a connection of the construction to the anterior section of capsular bag in the eye. For example, the flange can be adapted to be positioned under the rim of the capsullorhexis in the capsular bag, meaning: in between the anterior capsular bag and any optical element inside the capsular bag.

Said flange can be of another material as the material of which the accommodating intraocular lens construction is made. For example, the construction can be made of any flexible acrylate material, while the flange can be made of, for example, sturdy PMMA material or a metal fixed to the construction, by, for example, a pin-in-hole connection.

So, the accommodating intraocular lens is an optical addition to any optical element in the capsular bag, additional to, for example, the natural lens of the eye, or, alternatively, to any artificial lens implanted in the bag prior to implantation of the accommodating intraocular lens.

Such accommodating intraocular lens construction can also include at least one additional optical surface fitted to at least one optical surface, for example, a spherical optical surface adapted to provide correction of the refraction of the eye, or, alternatively, a toric optical surface adapted to provide correction of astigmatism of the eye, or, alternatively, any combination of additional surfaces adapted to provide correction of any combination of aberrations of the eye.

Such accommodating intraocular lens construction can be firmly coupled to any optical element in the capsular bag, for example, by a pin-in-hole system with the optical element in the capsular bag being any artificial lens implanted prior to implantation of the accommodating intraocular lens construction.

So, a lens combination according can comprise at least one free-form surface shaped according to a Zernike polynomial of which the order exceeds any third order Zernike polynomial.

Such lens combination can comprise a combination of at least two free-form surfaces each shaped according to a Zernike polynomial of which the order exceeds the third order Zernike polynomial with the combination to provide a cubic, a third Zernike order, modulation of the wavefront of the incoming light. Alternatively, the lens combination can comprise a combination of at least two free-form surfaces each shaped according to a Zernike polynomial of the third order, a cubic shape, which cubic shape also comprises at least one additional free-form shape according Zernike polynomial exceeding the third order Zernike polynomial which additional free-form shapes, in combination, are adapted to provide at least one high order variable modulation of any order of the incoming light beam. Such variable modulation can be, for example variable modulation to correct for variable trefoil aberration of the eye.

All documents and their illustrations referred to in this document are considered to be incorporated in this document.

1. An accommodative intraocular lens combination with the lens combination comprising at least two lens sections comprising at least one fixed power lens section adapted to provide fixed optical power to the eye with the section comprising at least one fixed optical power lens fitted with at least one fixed power lens mechanical construction and with the combination comprising at least one variable power lens section adapted to provide variable optical power to the eye with the section comprising at least one variable power lens fitted with at least one variable power lens mechanical construction wherein the fixed power lens section and the variable power lens section are, optically and mechanically, fully independent.

2. A lens combination according to claim 1, wherein the lens combination comprises a fixed power lens mechanical construction which comprises at least one positioning means adapted to provide positioning, anchoring, of only the fixed optical power lens into the eye.

3. The lens combination according to claim 1, wherein the lens combination comprises a variable lens mechanical construction which comprises at least one positioning means adapted to provide positioning, anchoring, of only the variable optical lens into the eye.

4. The lens combination according to claim 1, wherein the lens combination comprises variable lens mechanical construction which comprises at least one movement transfer means which component is adapted to provide transfer of movement of at least one driving component in the eye to at least one component of only the variable power lens.

5. The lens combination according to claim 3, wherein the lens combination comprises a variable power lens mechanical construction which comprises at least one combined means which means is adapted to provide both positioning, anchoring, of the variable lens section in the eye and is adapted to provide transfer of movement of at least one driving component in the eye to at least one component of only the variable power lens.

6. The lens combination according to claim 3, wherein the lens combination comprises a variable power lens mechanical construction which comprises a transfer means adapted to provide translation of movement of at least one driving means in the eye into movement of at least one component of only the variable power lens in a direction parallel to the optical axis.

7. The lens combination according to claim 3, wherein the lens combination comprises a variable power lens mechanical construction which comprises at least one transfer means adapted to provide translation of movement of at least one driving component in the eye into movement of at least one component of only the variable power lens in a direction perpendicular to the optical axis.

8. The lens combination according to claim 1, wherein the lens combination comprises a variable power lens which comprises at least one combination of at least two optical elements each fitted with at least one free-form optical surface with the combination of the free-form optical surfaces adapted to provide a lens of variable optical power of which the degree of optical power depends on the degree of mutual translation of the free-form optical surfaces.

9. The lens combination according to claim 1, wherein the lens combination comprises a variable power lens which comprises at least one elastic optical lens which lens is adapted to provide variable optical power by gradual curvature change of at least one optical surface of the variable lens.

10. The lens combination according to claim 1, wherein the lens combination comprises at least one additional optical surface adapted to provide optical correction of at least one fixed residual optical aberration of the eye.

11. The lens combination according to claim 1, wherein the lens combination comprises a variable power lens section which comprises at least one additional optical surface adapted to provide optical correction of at least one variable residual optical aberration of the eye.

12. The lens combination according to claim 1, wherein the lens combination comprises a variable power lens section which comprises at least one movement transfer component adapted to provide transfer of movement from any at least one driving means in the eye to the variable power lens.

13. The lens combination according to claim 12, wherein the driving means is at least one natural component of the eye.

14. The lens combination according to claim 12, wherein the driving means is at least one artificial component in the eye.

15. The lens combination according to claim 1, wherein the lens comprises a fixed power lens section positioned inside the capsular bag of the eye and a variable power lens section positioned at the sulcus plane.

16. The lens combination according to claim 1, wherein the lens combination comprises at least one free-form surface shaped according to a Zernike polynomial of which the order exceeds any third order Zernike polynomial.

17. The lens combination according to claim 16, wherein the lens combination comprises a combination of at least two free-form surfaces each shaped according to a Zernike polynomial of which the order exceeds the third order Zernike polynomial with the combination is adapted to provide a cubic, a third Zernike order, modulation of the wavefront of the incoming light.

18. The lens combination according to claim 16, wherein the lens combination comprises a combination of at least two free-form surfaces each shaped according to a Zernike polynomial of the third order, a cubic shape, which cubic shape comprises also at least two additional free-form shape according Zernike polynomial exceeding the third order Zernike which additional free-form shapes, in combination, are adapted to provide any, at least one, high order variable modulation of any order of the incoming light beam.

19. A method for implanting a lens combination according to claim 1, into an eye wherein the method comprises the following steps:
removing the natural lens from the eye
implanting the fixed power lens section into the eye
evaluating any undesired residual fixed and variable aberrations of the eye
implanting of the variable power lens section into the eye which variable power lens section is also adapted to also provide to the eye with optical correction of at least one undesired residual optical aberrations of the eye implanted with only the fixed power lens section.

20. The method for implanting a lens combination according to claim 1, into an eye wherein the method comprises the following steps:
removing the natural lens from the eye
implanting the fixed power lens section and the variable power lens section into the eye into the eye.

21. The method for implanting an accommodative intraocular lens according to claim 1, into an eye wherein the method comprises the following steps:
evaluating of any undesired aberrations of eye
implanting of the variable power lens section into the eye with the variable power lens section being adapted to provide accommodation to the eye and to provide optical correction of any number of undesired optical aberrations.

22. The method for implanting a lens combination, or specific section thereof, according to claim 18 into an eye wherein the eye is a phakic eye.

23. The method for implanting a lens combination according to claim 18 into an eye wherein the eye is an aphakic eye.

24. The method for implanting a lens combination, or specific section thereof, according to claim 18 into an eye wherein the eye is an pseudophakic eye.
